(19) United States
(12) Patent Application Publication
(10) Pub. No.: US 2014/0128484 A1
(43) Pub. Date: May 8, 2014

(54) CONVERTING NATURAL GAS TO ORGANIC COMPOUNDS

(71) Applicant: H R D Corporation, Sugar Land, TX (US)

(72) Inventors: Abbas Hassan, Sugar Land, TX (US); Aziz Hassan, Sugar Land, TX (US); Rayford G. Anthony, College Station, TX (US); Gregory G. Borsinger, Chatham, NJ (US)

(73) Assignee: H R D Corporation, Sugar Land, TX (US)

(21) Appl. No.: 14/071,330

(22) Filed: Nov. 4, 2013

Related U.S. Application Data
(60) Provisional application No. 61/723,219, filed on Nov. 6, 2012.

(51) Int. Cl. B01J 23/89; C07C 2/84; B01J 19/28; B01J 27/24; B01J 19/00

(52) U.S. Cl. CPC B01J 23/8993; B01J 27/24; B01J 19/0073; B01J 19/28; C07C 2/84
USPC 518/704; 502/100; 502/303; 502/200; 422/129; 422/209; 422/211; 585/658

(57) ABSTRACT

Herein disclosed is a catalyst composition for producing organic compounds comprising (a) a catalyst that promotes the oxidative coupling of methane (OCM) and a methane steam reforming (MSR) catalyst, wherein the catalyst composition causes oxidative dehydrogenation to form reactive species and oligomerization of the reactive species to produce the organic compounds; or (b) a catalyst that promotes syngas generation (SG) and a Fischer-Tropsch (FT) catalyst wherein the catalyst composition causes non-oxidative dehydrogenation to form reactive species and oligomerization of the reactive species to produce the organic compounds; or (c) a SG catalyst, a MSR catalyst, and a FT catalyst wherein the catalyst composition causes non-oxidative dehydrogenation to form reactive species and oligomerization of the reactive species to produce the organic compounds; or (d) a FT catalyst and a MSR catalyst wherein the catalyst composition causes reforming reactions and chain growing reactions to produce the organic compounds.

### CROSS-REFERENCE TO RELATED APPLICATIONS

This application claims the benefit under 35 U.S.C. §119(e) of U.S. Provisional Patent application No. 61/723,219 filed Nov. 6, 2012, the disclosure of which is hereby incorporated herein by reference.

### STATEMENT REGARDING FEDERALLY SPONSORED RESEARCH OR DEVELOPMENT

Not Applicable.

### FIELD OF THE INVENTION

The present invention relates generally to converting natural gas to organic compounds. More particularly, the present invention relates to utilizing novel catalysts to convert natural gas to more valuable organic compounds.

### BACKGROUND

Natural gas, consisting primarily of methane, is an important fuel source. Natural gas also contains alkanes such as ethane, propane, butanes, and pentanes. Alkanes of increasing carbon number are normally present in decreasing amounts in crude natural gas. Carbon dioxide, nitrogen, and other gases may also be present. Most natural gas is situated in areas that are geographically remote from population and industrial centers. It is often difficult to utilize natural gas as an energy resource because of the costs and hazards associated with compression, transportation, and storage of natural gas.

Various efforts have been made to convert natural gas (primarily methane) to organic carbon compounds, including liquid hydrocarbons and alcohols such as methanol. For example, one method is a two-step conversion process. In the first step, methane is reformed with water vapor (also called steam reforming) to produce carbon monoxide and hydrogen (i.e., synthesis gas or “syngas”):

CH₄+H₂O→CO+3H₂

In a second step, the produced syngas is converted to hydrocarbons. For instance, Sasol Ltd. of South Africa utilizes the Fischer-Tropsch process and utilizes both natural gas and coal feedstock to provide fuels that boil in the middle distillate range. Middle distillates may be defined as organic compounds that are produced between the kerosene and lubricating oil fractions in the refining processes. Middle distillates include light fuel oils and diesel fuel as well as hydrocarbon waxes.

It is also possible to convert natural gas to syngas via catalytic partial oxidation. In this process, natural gas is mixed with air, oxygen-enriched air, or oxygen, and introduced to a catalyst at elevated temperatures and pressures. The partial oxidation of methane yields a syngas mixture with a H₂:CO molar ratio of 2:1, as shown below:

CH₄+½O₂→CO+2H₂

The partial oxidation reaction is exothermic and requires the catalyst to be in the oxidative state, while the steam reforming reaction is strongly endothermic and requires the catalyst to be in a reducing state. Because the partial oxidation reaction is exothermic, it is difficult to control the reaction temperature in the catalyst bed. This is particularly true when scaling up the reaction from a micro reactor (e.g., ¼ in (about 6 mm) diameter reactor tube and less than 1 gram of catalyst) to a larger scale commercial reactor unit. This is because of the additional heat generated in large reactors relative to the limited heat transfer area available. If heat is not removed such that temperature control may be maintained, partial oxidation may transition to full oxidation, with the major quantity of end products being relatively low value carbon dioxide and water instead of syngas.

It is predicted that natural gas will outlast oil reserves by a significant margin and large quantities of natural gas are available in many areas worldwide. Therefore, there is continuing need and interest in developing methods to convert natural gas to organic compounds in an economical fashion to better utilize this resource.

### SUMMARY

Herein disclosed is a catalyst composition for producing organic compounds comprising (a) a catalyst that promotes the oxidative coupling of methane (OCM) and a methane steam reforming (MSR) catalyst, wherein the catalyst composition causes oxidative dehydrogenation to form reactive species and oligomerization of the reactive species to produce the organic compounds; or (b) a catalyst that promotes syngas generation (SG) and a Fischer-Tropsch (FT) catalyst wherein the catalyst composition causes non-oxidative dehydrogenation to form reactive species and oligomerization of the reactive species to produce the organic compounds; or (c) a SG catalyst, a MSR catalyst, and a FT catalyst wherein the catalyst composition causes non-oxidative dehydrogenation to form reactive species and oligomerization of the reactive species to produce the organic compounds; or (d) a FT catalyst and a MSR catalyst wherein the catalyst composition causes reforming reactions and chain growing reactions to produce the organic compounds.

In an embodiment, the OCM catalyst comprises a transition metal. In an embodiment, the OCM catalyst comprises a rare earth metal oxide. In an embodiment, the OCM catalyst comprises a component selected from the group consisting of sodium oxide, cobalt oxide, tungsten oxide, silicon oxide, manganese oxide, and combinations thereof. In an embodiment, the OCM catalyst comprises silicon nitride.

In an embodiment, the MSR catalyst comprises a metal oxide wherein the metal is selected from the group consisting of cobalt (Co), iron (Fe), molybdenum (Mo), tungsten (W), cerium (Ce), rhodium (Rh), platinum (Pt), palladium (Pd), titanium (Ti), zinc (Zn), nickel (Ni), ruthenium (Ru), and combinations thereof. In an embodiment, the MSR catalyst comprises a metal compound, wherein the metal is selected from the group consisting of nickel (Ni), cobalt (Co), iron (Fe), ruthenium (Ru), molybdenum (Mo), tungsten (W), rhodium (Rh), and combinations thereof. In an embodiment, the MSR catalyst comprises a support material selected from the group consisting of alumina, silica, magnesia, and combinations thereof.

In an embodiment, the weight ratio of the OCM catalyst to the MSR catalyst in catalyst composition (a) is in the range of from about 50:1 to about 99:1.

In an embodiment, the catalysts are deposited on a support to form the catalyst composition.

In an embodiment, the catalysts are dry blended to form the catalyst composition. In an embodiment, the catalyst composition further comprises a promoter. In an embodiment, the catalyst composition maintains its catalytic activity in the temperature range of from about 300° C. to about 1200° C.

In an embodiment, catalyst composition (a) comprises 0.1-99 wt % of rhodium.

Also disclosed herein is a method of preparing a catalyst composition, comprising dry blending (a) a catalyst that promotes the oxidative coupling of methane (OCM) and a methane steam reforming (MSR) catalyst, wherein the catalyst composition causes oxidative dehydrogenation to form reactive species and oligomerization of the reactive species to produce the organic compounds; or (b) a catalyst that promotes syngas generation (SG) and a Fischer-Tropsch (FT) catalyst wherein the catalyst composition causes non-oxidative dehydrogenation to form reactive species and oligomerization of the reactive species to produce the organic compounds; or (c) a SG catalyst, a MSR catalyst, and a FT catalyst wherein the catalyst composition causes non-oxidative dehydrogenation to form reactive species and oligomerization of the reactive species to produce the organic compounds; or (d) a FT catalyst and a MSR catalyst wherein the catalyst composition causes reforming reactions and chain growing reactions to produce the organic compounds.

Disclosed is another method of preparing a catalyst composition, comprising depositing on an inert support: (a) a catalyst that promotes the oxidative coupling of methane (OCM) and a methane steam reforming (MSR) catalyst, wherein the catalyst composition causes oxidative dehydrogenation to form reactive species and oligomerization of the reactive species to produce the organic compounds; or (b) a catalyst that promotes syngas generation (SG) and a Fischer-Tropsch (FT) catalyst wherein the catalyst composition causes non-oxidative dehydrogenation to form reactive species and oligomerization of the reactive species to produce the organic compounds; or (c) a SG catalyst, a MSR catalyst, and a FT catalyst wherein the catalyst composition causes non-oxidative dehydrogenation to form reactive species and oligomerization of the reactive species to produce the organic compounds; or (d) a FT catalyst and a MSR catalyst wherein the catalyst composition causes reforming reactions and chain growing reactions to produce the organic compounds.

In an embodiment, the inert support is selected from the group consisting of alumina, zeolite, zirconia, silica, glass, magnesia, a metal, a metal oxide, and combinations thereof.

Further disclosed herein is a method of producing organic compounds, comprising sizing a catalyst composition, the catalyst composition comprising (a) a catalyst that promotes the oxidative coupling of methane (OCM) and a methane steam reforming (MSR) catalyst, wherein the catalyst composition causes oxidative dehydrogenation to form reactive species and oligomerization of the reactive species to produce the organic compounds; or (b) a catalyst that promotes syngas generation (SG) and a Fischer-Tropsch (FT) catalyst wherein the catalyst composition causes non-oxidative dehydrogenation to form reactive species and oligomerization of the reactive species to produce the organic compounds; or (c) a SG catalyst, a MSR catalyst, and a FT catalyst wherein the catalyst composition causes non-oxidative dehydrogenation to form reactive species and oligomerization of the reactive species to produce the organic compounds; or (d) a FT catalyst and a MSR catalyst wherein the catalyst composition causes reforming reactions and chain growing reactions to produce the organic compounds to a size suitable for use in a reactor; adding a quantity of the sized catalyst composition to the reactor; contacting a feed gas stream comprising natural gas and steam and optionally hydrogen in the reactor in the presence of the catalyst composition; and collecting the effluent from the reactor, wherein the effluent comprises organic compounds.

Yet disclosed further is a method of producing organic compounds comprising contacting a reactant gas mixture comprising natural gas and steam and optionally hydrogen with a catalyst composition, wherein the catalyst composition comprises (a) a catalyst that promotes the oxidative coupling of methane (OCM) and a methane steam reforming (MSR) catalyst, wherein the catalyst composition causes oxidative dehydrogenation to form reactive species and oligomerization of the reactive species to produce the organic compounds; or (b) a catalyst that promotes syngas generation (SG) and a Fischer-Tropsch (FT) catalyst wherein the catalyst composition causes non-oxidative dehydrogenation to form reactive species and oligomerization of the reactive species to produce the organic compounds; or (c) a SG catalyst, a MSR catalyst, and a FT catalyst wherein the catalyst composition causes non-oxidative dehydrogenation to form reactive species and oligomerization of the reactive species to produce the organic compounds; or (d) a FT catalyst and a MSR catalyst wherein the catalyst composition causes reforming reactions and chain growing reactions to produce the organic compounds.

In an embodiment, contacting the reactant mixture with catalyst composition (a) takes place in an oxidizing environment. In an embodiment, the MSR catalyst of catalyst composition (a) further comprises metals that promote FT reactions, wherein the reactant mixture is contacted with catalyst composition (a) in an oxidative atmosphere and a reducing atmosphere in an alternating fashion.

In an embodiment, no hydrogen or carbon oxides are added.

In an embodiment, the method comprises adding hydrogen or carbon oxides to the catalytic reaction.

In an embodiment, the hydrogen addition reduces metal oxides to metal to activate the catalyst composition.

In an embodiment, contacting the reactant gas mixture with the catalyst composition takes place in a reactor selected from the group consisting of fluidized bed reactor, fixed-bed reactor, bubble column, totally mixed slurry reactor, back mixed flow reactor, membrane reactor, radial flow reactor, tube and shell reactor, and multiple reactors in series with inter-stage feeds.

In an embodiment, contacting the reactant gas mixture with the catalyst composition takes place at a temperature in the range of from about 300° C. to about 1200° C. In an embodiment, contacting the reactant gas mixture with the catalyst composition takes place at a pressure in the range of from about 0.1 atm to about 100 atm. In an embodiment, the molar ratio of steam to natural gas in the reactant gas mixture is in the range of from about 200 to about 1. In an embodiment, the single pass yield of organic compounds is no less than 75%.

Disclosed herein is a method of producing organic compounds comprising contacting a reactant gas mixture comprising natural gas and steam and optionally hydrogen with a catalyst composition to form organic compounds, wherein the steam is preheated; and wherein the catalyst composition comprises (a) a catalyst that promotes the oxidative coupling of methane (OCM) and a methane steam reforming (MSR) catalyst, wherein the catalyst composition causes oxidative dehydrogenation to form reactive species and oligomerization of the reactive species to produce the organic compounds; or (b) a catalyst that promotes syngas generation (SG) and a Fischer-Tropsch (FT) catalyst wherein the catalyst composition causes non-oxidative dehydrogenation to form reactive species and oligomerization of the reactive species to produce the organic compounds; or (c) a SG catalyst, a MSR catalyst, and a FT catalyst wherein the catalyst composition causes non-oxidative dehydrogenation to form reactive species and oligomerization of the reactive species to produce the organic compounds; or (d) a FT catalyst and a MSR catalyst wherein the catalyst composition causes reforming reactions and chain growing reactions to produce the organic compounds.

In an embodiment, steam is distributed over the catalyst composition through a permeable membrane. In an embodiment, the reactant gas mixture is distributed over the catalyst composition through a sintered metal tube.

Also described herein is a gas exchanger comprising a gas seal separating the exchanger into at least two compartments wherein the seal prevents gas exchange between the compartments; and a catalyst.

In an embodiment, the gas exchanger is a rotary gas exchanger. In an embodiment, the rotary gas exchanger is driven by a mechanical drive configured to rotate the exchanger in either a clockwise or counterclockwise direction. In an embodiment, the gas seal is configured to withstand a temperature of up to 900° C. In an embodiment, the gas exchanger further comprises an outer shell, configured to contain the catalyst.

In an embodiment, the catalyst is in a bed formation. In an embodiment, the catalyst bed is configured to cause turbulent gas flow and to provide minimal pressure drop across the catalyst bed. In an embodiment, the catalyst is coated on ceramic or metal surfaces used to pack the exchanger.

Also described herein is a method comprising converting a reactant into a product in a rotating reactor, exchanging heat between the reactant and the product, promoting the reaction with a catalyst, and re-activating the catalyst; wherein the reactor comprises a rotary gas heat exchanger comprising the catalyst and a gas seal separating the exchanger into at least two compartments, and wherein the seal prevents gas exchange between the compartments.

In an embodiment, the reactor comprises an individual rotary gas heat exchanger. In an embodiment, the reactor comprises multiple stacked rotary gas heat exchangers.

In an embodiment, the method further comprises cooling a gas by inter-exchanger heat transfer or gas injection. In an embodiment, the method further comprises inter-exchanger gas injection.

In an embodiment, the method further comprises rotating the reactor at a speed sufficient to provide sufficient residence time for the reactant to be converted into the product. In an embodiment, the method further comprises rotating the reactor at a speed sufficient to provide sufficient residence time for the catalyst to be re-activated.

The foregoing has outlined rather broadly the features and technical advantages of the invention in order that the detailed description of the invention that follows may be better understood. Additional features and advantages of the invention will be described that form the subject of the claims of the invention. It should be appreciated by those skilled in the art that the conception and the specific embodiments disclosed may be readily utilized as a basis for modifying or designing other structures to accomplish the same purposes of the invention. It should also be realized by those skilled in the art that such equivalent constructions do not depart from the spirit and scope of the invention as set forth in the appended claims.

### BRIEF DESCRIPTION OF THE DRAWINGS

For a more detailed description of the preferred embodiments of the present disclosure, reference will now be made to the accompanying drawings, wherein:

FIG. 1 illustrates a rotary gas exchanger suitable for the disclosed method and system in accordance with an embodiment of this disclosure.

FIGS. 2a and 2b illustrate a porous membrane reactor where the catalyst is coated onto a porous membrane that provides for uniform distribution of feed gases across the catalyst bed. FIG. 2b illustrates the porous membrane structure of FIG. 2a.

FIGS. 3a, 3b, 4a, and 4b illustrate alternative reactor designs in accordance with an embodiment of this disclosure.

FIGS. 5a-5e illustrate a reactor assembly in accordance with embodiments of this disclosure. FIG. 5a illustrates an exemplary placement of a catalyst in accordance with an embodiment of this disclosure. FIG. 5b illustrates exemplary placement of graphite ballast in accordance with an embodiment of this disclosure. FIG. 5c illustrates the relative locations of an exemplary preheat section and an exemplary reactor in accordance with embodiments of this disclosure. FIG. 5d illustrates cross-sectional views of exemplary reactor designs in accordance with embodiments of this disclosure. FIG. 5e illustrates an exemplary exploded assembly view of components in accordance with embodiments of this disclosure.

FIG. 6 illustrates a gas distribution device (sparger) suitable for use with the methods and systems described in accordance with embodiments of this disclosure.

FIGS. 7a-d are energy-dispersive X-ray spectra (EDS) of a sample catalyst prepared according to Example 2. More details are provided in Example 3.

FIGS. 8a and 8b are scanning electron micrographs of the same catalyst prepared according to Example 2.

### NOTATION AND NOMENCLATURE

For nomenclature purposes, reference to Groups from the Periodic Table of the Elements refers to the IUPAC Periodic Table of Elements (Jun. 22, 2007 version) published by the International Union of Pure and Applied Chemistry (IUPAC).

In this disclosure, a syngas generating catalyst, which is abbreviated as SG catalyst for ease of reference. A Fischer-Tropsch catalyst is abbreviated as FT catalyst for ease of reference. A methane steam reforming catalyst is abbreviated as MSR catalyst for ease of reference, which is equivalent to a steam methane reforming catalyst or a steam reforming catalyst. A catalyst that promotes oxidative coupling of methane is abbreviated as OCM catalyst for ease of reference. In this disclosure, the use of the terms SG, FT, MSR, and/or OCM catalysts serves the purpose of referring to these categories of catalysts but does not limit these catalysts in their function according to the conventional understanding of the art. The reactions that these catalysts promote or activate should be understood in the context of this disclosure.

As used herein, the term ‘sintered metal’ refers to powdered metal that is compressed and sintered.

In this disclosure, oxidative coupling of methane (OCM) includes both complete oxidation and partial oxidation of methane. In most cases, partial oxidation of methane occurs unless otherwise specified. During partial oxidation of methane, intermediate species, such as CH₃, CH₂, and CH, are generated, which participate in the formation of organic compounds.

In this disclosure, percentages for gases are volume based and percentages for solids are weight based unless otherwise specified.

Certain terms are used throughout the following description and claims to refer to particular system components. This document does not intend to distinguish between components that differ in name but not function.

In the following description and in the claims, the terms “including” and “comprising” are used in an open-ended fashion, and thus should be interpreted to mean “including, but not limited to.”.

### DETAILED DESCRIPTION

Overview.

In an embodiment, a method of producing organic compounds comprises contacting a reactant gas mixture comprising natural gas and steam with a Multifunctional (MF) Catalyst. In some embodiments, the feed includes hydrogen. In some embodiments, the feed also includes carbon oxides and hydrogen. In some embodiments, oxygen or air is included in the feed.

In some embodiments, the MF catalyst comprises a MSR catalyst and a FT catalyst. In some embodiments, the MF catalyst comprises a SG catalyst, a MSR catalyst, and a FT catalyst. In some embodiments, the MF catalyst comprises a SG catalyst and a FT catalyst. In some embodiments, the MF catalyst comprises an OCM catalyst and a MSR catalyst.

In this disclosure, the formation of organic compounds is initiated by the removal of one or more hydrogen's (H) from methane (CH₄), thereby creating reactive species (e.g., CH₃, CH₂, and/or CH). The reactive species then combine to form organic compounds (e.g., methanol, C2+ compounds). In some cases, dehydrogenation (hydrogen removal) from methane involves the use of oxygen, which is termed oxidative dehydrogenation herein. In some cases, dehydrogenation (hydrogen removal) from methane does not involve the use of oxygen, which is termed non-oxidative dehydrogenation herein. For example, oxidative dehydrogenation reactions may utilize metal catalysts such as OCM catalysts; and non-oxidative dehydrogenation reactions may utilize metals in MSR catalysts.

Herein various catalytic reactions are cited such as OCM, FT, MSR, and SG. Various metals, metal combinations and metal states (i.e. oxides) are noted to promote these reactions. In certain cases, a metal is used under different conditions and/or in different states to promote different reactions. For example, nickel in its oxidized state is used as a promoter of oxidation of hydrocarbons; nickel in its reduced state is used as a hydrogenation catalyst in the presence of hydrogen and unsaturated hydrocarbons. Reduced nickel is also used in methane steam reforming (CH₄+H₂O→CO+3H₂).

In an embodiment, a reactor design (e.g., rotating catalytic reactor) is described that significantly reduces undesirable carbon oxide formation. In various embodiments, other types of reactor designs are described

MSR Catalysts.

In an embodiment, the MSR catalyst of this disclosure comprises a metal selected from the group consisting of cobalt (Co), iron (Fe), molybdenum (Mo), tungsten (W), cerium (Ce), rhodium (Rh), platinum (Pt), palladium (Pd), titanium (Ti), zinc (Zn), nickel (Ni), ruthenium (Ru), and combinations thereof. In an embodiment, the MSR catalyst comprises rhodium (Rh) catalysts, nickel (Ni) catalysts, ruthenium (Ru) catalysts, platinum (Pt) catalysts, or palladium (Pd) catalysts. Examples of rhodium catalysts include rhodium coated α-Al₂O₃ foam monoliths and Ce—ZrO₂-supported Rh catalyst. Examples of nickel catalysts include unsupported nickel powder catalysts, ceramic-supported nickel catalysts, Ce—ZrO₂-supported Ni catalysts, doped ceria supported Ni—Cu catalyst, and α-Al₂O₃-supported nickel catalyst. Examples of ruthenium catalysts include Ru-added to Ni catalysts supported on Al₂O₃, La₂O₃, MgO, or MgAl₂O₄, and bimetallic catalysts comprising Ru and Ni. Examples of platinum catalysts include Pt/Al₂O₃, Pt/ZrO₂ and Pt/CeO₂ catalysts prepared, for example, by incipient wetness impregnation of calcined γ-alumina (Engelhard Corporation Catalyst), zirconium hydroxide (MEL Chemicals), and cerium ammonium nitrate (Aldrich) supports. Examples of palladium catalysts include alumina supported palladium catalysts and Pd/ZnO catalysts prepared by impregnation or micro-emulsion techniques. Other MSR catalysts include those disclosed in U.S. Pat. Nos. 7,670,987, 7,687,051, 7,687,050, 7,470,648, 7,569,511, and 6,355,589, the disclosures of which are hereby incorporated herein by reference for all purposes. A general treatment after the synthesis of a reforming catalyst is calcination (heating the sample in air, in order to clean up and stabilize the catalyst) and/or reduction of the catalyst (heating the sample in a reducing atmosphere containing hydrogen, in order to activate the catalytic metal). It is within the scope of this disclosure to utilize a MSR catalyst as known to one skilled in the art to form a MF catalyst as described herein.

SG Catalysts.

In an embodiment, the SG catalyst of this disclosure includes various oxides, halides and carbonates of both alkali and alkaline earth metals, transition metals, and combinations thereof. In an embodiment, a SG catalyst produces syngas from methane and carbon dioxide.

In an embodiment, the SG catalysts of this disclosure refer to those catalysts that produce/generate syngas other than MSR catalysts, for example, a metal-based catalyst that generates syngas from a carbon source (e.g., biomass). Such a SG catalyst contains a transition metal or noble metal, in combination with a lanthanide. In some cases, the lanthanide is cerium or lanthanum. In some cases, such a SG catalyst comprises Ni, Pd, Pt, Co, Rh, Ir, Fe, Ru, Os, Cu, Ag, Au, Re, or a combination thereof. In some cases, the metal-based SG catalyst is a rhodium-cerium catalyst. Further details of such SG catalysts are in US Patent Publication Nos. 20100200810 and 20110047864. It is within the scope of this disclosure to utilize a SG catalyst as known to one skilled in the art to form a MF catalyst as described herein.

FT catalysts. In embodiments, Fischer-Tropsch (FT) catalyst of this disclosure includes a Group VIII transition metal. Such transition metals include cobalt, iron, and ruthenium. In an embodiment, the FT catalyst comprises cobalt as the active component to promote the conversion reactions. In some cases, the FT catalyst also contains one or more noble metal promoters. The FT catalyst is able to produce organic compounds (e.g., C₂H₆, CₙH₂ₙ₊₂, n=2, 3, 4, . . . or higher numbers, or CₙH₂ₙ, n=2, 3, 4 . . . ) from syngas. A variety of catalysts may be used for the FT process, but the most common are the transition metals cobalt, iron, and ruthenium. In some cases, Nickel is used, but tends to favor methane formation (“methanation”). In an embodiment, the FT catalyst of this disclosure includes those for iso-synthesis, e.g. formation of iso-paraffins, and iso-olefins, such as (1) ZnO—Al₂O₃, (2) Al₂O₃, (3) ThO₂, (4) ZnO with ThO₂ or ZrO₂, (5) ThO₂—Al₂O₃. It is within the scope of this disclosure to utilize a FT catalyst as known to one skilled in the art to form a MF catalyst as described herein.

OCM Catalysts.

In an embodiment, the OCM catalyst comprises a transition metal. In an embodiment, the OCM catalyst comprises an alkali metal. In an embodiment, the alkali metal is selected from the group consisting of lithium, sodium, potassium, rubidium, cesium, and mixtures thereof.

In an embodiment, the OCM catalyst comprises a Group 2 metal. In an embodiment, the Group 2 metal is selected from the group consisting of strontium, calcium, barium, and magnesium.

In an embodiment, the OCM catalyst comprises a component selected from the group consisting of sodium oxide, cobalt oxide, tungsten oxide, silicon oxide, manganese oxide, and combinations thereof. In an embodiment, the OCM catalyst comprises silicon nitride. In an embodiment, the OCM catalyst is a supported catalyst. In various embodiments, the support is an inert material having high surface area. In various embodiments, the OCM catalyst comprises a promoter.

In an embodiment, the OCM catalyst is a nickel-based catalyst. In an embodiment, the OCM catalyst is a cobalt-based catalyst. In an embodiment, the OCM catalyst comprises magnesium, manganese, or combination thereof. In an embodiment, the OCM catalyst comprises oxides of magnesium, oxides of manganese, or combinations thereof.

In an embodiment, the OCM catalyst comprises an alkali metal oxide. In an embodiment, the OCM catalyst comprises a rare earth metal oxide.

Other examples of the OCM catalyst and preparation methods may be found in U.S. Pat. Nos. 7,291,321; 7,250,543; 6,096,934; 5,877,387; 5,849,973; 5,736,107; 5,599,510; 5,321,185; 5,132,482; 5,132,481; 5,118,654; 5,097,086; 5,066,629; 4,956,327; 4,945,078; 4,935,572; and 4,826,796, the disclosures of which are hereby incorporated herein by reference for all purposes. It is within the scope of this disclosure to utilize an OCM catalyst as known to one skilled in the art to form a MF catalyst as described herein.

Other MF Catalysts.

In a further embodiment, a MF catalyst comprises oxide(s) of Zn, oxide(s) of Mn, oxide(s) of Co, oxide(s) of Ni, oxide(s) of Mg, or oxide(s) of Fe. When this catalyst is contacted with steam and methane, the overall reaction is:

13CH₄+14H₂O→C₂H₄+4CO+5CO2+36H₂.

In an embodiment, steam and methane are reacted under the action of such MF catalyst at a temperature of about 900° C.

Formation of MF Catalyst.

In an embodiment the MF catalysts of this disclosure are fabricated utilizing combinations of powdered metal oxides and/or metal salts that promote two or more reactions encompassing SG, MSR, FT, and OCM reactions. Selection of the type metal to promote each reaction is determined by the reactivity and selectivity of the metal to produce the desired reaction product but also by the ability of the metal to withstand the operating conditions required to produce the desired reaction products without melting or sintering.

In an embodiment, a MF catalyst is formed by dry blending a powder MSR catalyst and a powder FT catalyst. In an embodiment, a MF catalyst is formed by dry blending a powder MSR catalyst, a powder SG catalyst, and a powder FT catalyst. In an embodiment, a MF catalyst is formed by dry blending a powder SG catalyst and a powder FT catalyst. In an embodiment, a MF catalyst is formed by dry blending a powder MSR catalyst and a powder OCM catalyst.

In some embodiments, the MF catalyst is prepared by dry blending an OCM catalyst with an MSR catalyst. In some cases, the ratio between the OCM catalyst and the MSR catalyst is in the range of 50:1 to 99:1. In some cases, the OCM catalyst and the MSR catalyst are in the form of powder or ultra fine powder.

In some embodiments, the MF catalyst is prepared by depositing an OCM catalyst and an MSR catalyst on an inert support. The inert support may comprise, without limitation, alumina, zeolite, zirconia, silica, glass, magnesia, a metal, or a metal oxide. Other types of inert support are known in the art and within the scope of this disclosure. In some cases, the inert support comprises a high surface area substrate. In some cases, the inert support comprises a porous substrate. The use of high surface area substrate in a support increases catalytic activity. In some cases, the use of high surface area substrate enables the use of reduced metal content.

In some embodiments, a method of forming the MF catalyst comprises preparing an OCM catalyst; crushing the OCM catalyst; mixing the crushed OCM catalyst with an MSR catalyst to form a catalyst mixture; pelletizing the catalyst mixture to form catalyst pellets; crushing the catalyst pellets and annealing the crushed catalyst pellets at increasing temperatures with a predetermined temperature-time profile. In some cases, preparing the OCM catalyst comprises forming an aqueous slurry comprising an alkaline earth metal salt, a powdered metal salt, and a powdered transition metal oxide; adding a polymeric binder to the slurry to form a paste; drying the paste to form a powder; heating the powder at increasing temperatures at a predetermined temperature-time profile commensurate with the polymeric binder; and calcining the heated powder to form the OCM catalyst.

In some embodiments, the MF catalyst is further treated. For example, the MSR reaction requires the reforming metal in the catalyst to be reduced (metal and not oxides). Reduction of the steam reforming component of the MF catalyst may be by means of hydrogen at temperatures in excess of 180° C. The catalyst is reduced by passing a carrier gas such as nitrogen, natural gas, or steam through the catalyst and adding controlled amounts of hydrogen. For example, the catalyst is reduced in situ by heating to 180° C. for 4 h followed by 12 h at 230° C. in a gas mixture of 1% hydrogen/99% nitrogen (vol % or mol %). The activated catalyst is, however, pyrophoric. Upon exposure to air, the catalyst must be re-reduced and stabilized by surface oxidation. For steam-reforming, Ni or the noble metals Ru, Rh, Pd, Ir, Pt are used as the active metal in catalysts. Because of its low costs, Ni is the most widely used metal from this set. Ni, however, is less active than other of these metals.

For steam-reforming, Ni or the noble metals Ru, Rh, Pd, Ir, Pt are used as the active metal in catalysts. Because of its low costs, Ni is the most widely used metal from this set. Ni, however, is less active than other of these metals. These metals may be deposited on supports for methane reforming, which include alpha- and gama-Al₂O₃, MgO, MgAl₂O₄, SiO₂, ZrO₂, and TiO₂. In the case of methane reforming, promoters to inhibit carbon deposition on the active metal may be added. Suppression of carbon formation on (Ni-based) catalysts is achieved by adding small amounts of an alkali metal to the catalyst.

In an embodiment, the MF catalyst comprises a dry blend of an OCM catalyst and an MSR catalyst. In an embodiment, an OCM catalyst and an MSR catalyst are deposited on a support to form a MF catalyst. Under conventional operating conditions the Oxidative Coupling reaction requires the OCM component of the MF catalyst to be activated and the metals in the OCM catalyst component exist in an oxide form. Herein the MF catalyst is utilized in the reduced state to produce mainly syngas with some minor amounts of organic compounds produced.

In an embodiment, the content of OCM catalyst in the MF catalyst is in the range of from 91 wt % to 99 wt %, alternatively from 71 wt % to 89 wt %, alternatively from 50 wt % to 70 wt %, with the balance of the catalyst being an MSR catalyst. In an embodiment, the ratio between an OCM catalyst and an MSR catalyst in a MF catalyst is 99:1; alternatively 90:10; alternatively 80:20; or alternatively 70:30. In an embodiment, the weight ratio between an OCM catalyst and an MSR catalyst is in the range of from about 50:1 to about 99:1. In embodiments, silicon nitride is incorporated with an MSR catalyst if increasing the fusion temperature of the catalyst is desired.

In an embodiment, a MF catalyst comprises an OCM catalyst, wherein the OCM catalyst comprises a transition metal oxide, an alkali metal oxide, and an alkaline earth metal oxide; an MSR catalyst; a semimetal oxide; and a semimetal nitride. In some cases, the transition metal comprises cobalt or tungsten, the alkali metal comprises sodium, the alkaline earth metal comprises manganese, and the semimetal comprises silicon.

Without being limited by theory, the combination of metals or metal oxides in MF catalysts may also promote reduction of carbon dioxide, if present, in the presence of hydrogen. It is known that the presence of oxides of Group 3 and Group 4 elements in combination with transition metals of Groups 8, 9, and 10 may promote reduction of carbon dioxide in the presence of hydrogen. Further examples of such catalysts are listed in US Patent Application No. 20070149392 and U.S. Pat. Nos. 5,911,964 and 5,855,815, the disclosures of which are hereby incorporated herein by reference in their entirety for all purposes.

In an embodiment, a MF catalyst composition for producing syngas and minor amounts of organic carbon compounds when operated in a reducing atmosphere (e.g., absent of oxygen and present of hydrogen) comprises 0.1-99 wt % of rhodium. In some cases, the catalyst composition comprises 10-90 wt % of rhodium. In some cases, the catalyst composition comprises 20-80 wt % of rhodium. In some cases, the catalyst composition comprises 30-70 wt % of rhodium. In some cases, the catalyst composition comprises 40-60 wt % of rhodium. In some cases, the catalyst composition comprises more than 50 wt % of rhodium.

In some embodiments, addition of halogen by adding, for example, chlorine or a chlorine-containing compound further enhances catalyst life and selectivity to hydrocarbons. In some cases, halogen or halogen-containing compound is added to the mixture to give a final concentration ranging from about 0.001% volume/volume (“v/v”) to about 0.04% v/v. In other cases, halogen or halogen-containing compound is added to a final concentration ranging from about 0.008% v/v to about 0.02% v/v. Halogen may be introduced in any form to the catalyst composition.

Use of MF Catalyst.

In embodiments, the reaction temperature for utilizing the MF catalyst is in the range of from about 300° C. to about 1000° C.; alternatively from about 300° C. to about 900° C.; alternatively from about 350° C. to about 950° C. In embodiments, the reaction temperature for utilizing the MF catalyst is in the range of from about 400° C. to about 875° C.; or alternatively from about 400° C. to about 850° C.; or alternatively from about 450° C. to about 850° C. In some cases, the reaction temperature is in the range of from about 700° C. to about 900° C.; alternatively from about 750° C. to about 875° C.; or alternatively from about 775° C. to about 850° C.

In embodiments, the reaction pressure is in the range of from about 20 kPa to about 25,000 kPa; or alternatively from about 50 kPa to about 10,000 kPa; or alternatively from about 70 kPa to about 10,000 kPa. In an embodiment, the reaction pressure is in the range of from about 20 kPa to about 300 kPa.

In an embodiment wherein alcohols are produced, the reaction temperature is in the range of from about 300° C. to about 1200° C. In an embodiment, the reaction pressure is in the range of from about 0.1 atm to about 100 atm.

In an embodiment, the production of organic compounds comprises contacting a reactant gas mixture comprising natural gas and steam with optional addition of hydrogen and carbon oxides with a MF catalyst as described herein. In embodiments, the reactant gas may first be treated by means known to those skilled in the art to remove catalyst poisoning compounds such as sulfur-containing compounds.

In an embodiment, a method for producing an organic compound comprises contacting a reactant gas mixture comprising natural gas and steam with optional addition of hydrogen and carbon oxides with a catalytically effective amount of a MF catalyst. The reactant gas mixture may include other hydrocarbons such as, but not limited to, ethane, propane, butane, hexane, heptane, n-octane, iso-octane, naphthas, liquefied petroleum gas, and middle distillate hydrocarbons. In some embodiments, the feed gas includes steam. In some embodiments, the feed gas includes hydrogen. In some embodiment, the feed gas comprises at least about 50% methane by volume. In some embodiment, the feed gas comprises at least about 80% methane by volume. In certain embodiments, the feedstock is pre-heated before contacting the catalyst.

Operations.

The reactors as described herein may be arranged in series or in parallel to achieve desired yield and/or production throughput. In some embodiments, reactors of different designs are used in combination.

Under normal operation conditions, the feed gas consists primarily of methane and steam. In some cases, the methane composition ranges from 5% to 95% (mol %). In some cases, steam ranges from 1% to 95% (mol %).

In some embodiments, molecular oxygen is added to the feed gas when the MF catalyst becomes fouled. For example, O₂ acts in combination with the OCM catalyst to de-coke and regenerate the catalyst. In some embodiments, the feed gases are cycled between oxidative and reducing atmospheres. In an embodiment, the methane:oxygen:steam molar ratio ranges from about 1:1:1 to about 4:1:1. In an embodiment, the methane:oxygen:steam molar ratio ranges from about 1:1:1 to about 1:1:4. In an embodiment, the methane:oxygen:steam molar ratio ranges from about 10:1:10 to 1:1:10. In an embodiment, the methane:oxygen:steam molar ratio ranges from about 10:1:10 to 1:4:1.

In certain embodiments, the molar ratio of steam to natural gas in the feed is in the range of from about 1:1 to about 3:1; alternatively from about 5:1 to about 10:1; alternatively from about 10:1 to about 50:1; alternatively from about 200:1 to about 1:1.

In certain embodiments, the reactant gas mixture is passed over the catalyst at a space velocity of from about 200 to about 30,000 normal liters of gas per hour per liter of catalyst per hour (NL/L/h), alternatively from about 500 to 10,000 NL/L/h.

Some embodiments provide for retaining the catalyst in a fixed bed reaction zone. Any type of reactor may be used, such as, without limitation, fluidized bed reactors, fixed-bed reactors, bubble columns, totally mixed slurry reactors, back-mixed flow reactors, membrane reactors, radial flow reactors, and multiple reactors in series with inter-stage feeds.

An embodiment of the present invention utilizes reactors in series (either with or without inter-stage separation or addition of additional feed gases) and utilizes recycling of the unreacted and combustion by-products of the process to increase the overall yield of methane to organic compounds.

Rotary Gas Exchanger.

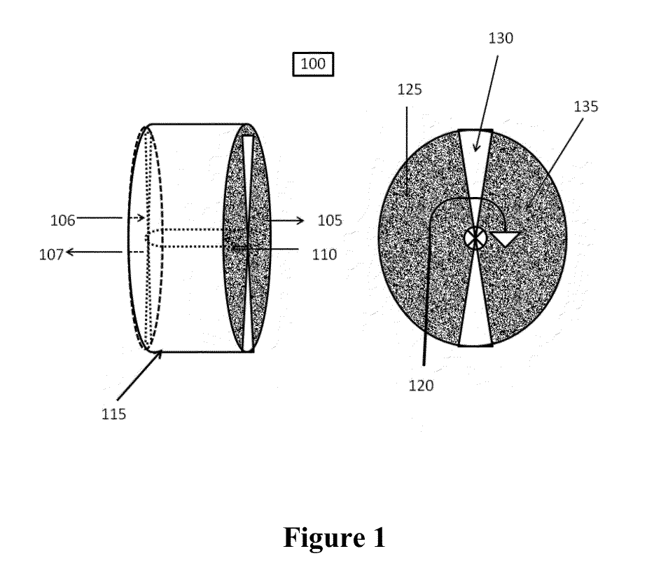

In an embodiment, a rotary gas exchanger (see FIG. 1) is utilized for the production of organic compounds. In this configuration the rotary gas exchanger 100 is split with a high temperature gas seal 130 that prevents gas exchange between the oxygen rich side 135 and the methane containing gas side 125. In some embodiments, the gas seal is configured to withstand a temperature of up to 900° C.

In embodiments, the oxygen rich side 135 may be air, oxygen or oxygen enriched air. A high temperature resistant outer shell 115 seals the catalyst bed within it. Oxygen rich gas 105 and 106 flows counter current to the methane rich gas 110. Within the rotary gas exchanger 100 is placed oxidative dehydrogenation catalyst (125 and 135) in a bed formation.

In embodiments, the rotary gas exchanger comprises a catalyst that utilizes oxygen to dehydrogenate methane, such as metal oxide catalyst used for OCM and methane either alone or in combination with metals used in steam reforming (MSR) catalyst. In embodiments, the oxygen rich gas 106 is an air stream that is preheated in an external heat exchanger (not shown) before it enters the rotary gas exchanger 100, thus recovering heat from the exiting reacted methane stream 107 that is rich in organic compounds. Catalyst may be in bead or pellet form of a size suitable to allow for desired gas flow. Catalyst may also be coated on a high surface area substrate such as alumna silicate. Alternatively, the catalyst is coated on high temperature ceramic or metal surfaces such as baffles or mesh that is used to pack the rotary gas exchanger 100. Suitable catalyst bed construction provides for turbulent gas flow and minimal pressure drop across the catalyst bed. The rotary gas exchanger 100 is driven by a mechanical drive 120 that rotates the exchanger in either a clockwise or counterclockwise direction at a speed that results in sufficient residence time for the catalyst 135 to become activated (oxygenated) when exposed to the oxygen rich side 106 to 105 of the gas flow.

Rotary gas exchangers may be used as individual exchangers or stacked to allow for multiple exchangers with inter-stage cooling or oxygen injection. Methane rich gas enters the exchanger 110 at a temperature and pressure suitable for the oxidative coupling reaction to occur. Preferably, the pressure differential between the oxygen rich 135 and methane rich 125 sections is minimized to prevent leakage across the high temperature seal 130. The methane 110 reacts with the oxidative coupling catalyst and exits the exchanger 107 with the formation of organic compounds and minimal undesirable side reactions such as CO and CO₂ formation due to the lack of free oxygen present. Once the catalyst is expended of oxygen, the catalyst is rotated into the oxygen rich section 135 of the rotary gas exchanger 100 where it once again becomes activated.

Since the oxidative dehydrogenation reaction is exothermic, the amount of catalyst used in each heat exchange element and the reactor configuration will be limited by the rise in temperature of the converted methane stream as it exits each exchanger. In some embodiments, an inert gas or steam may be used to help control temperatures.

Reactor with Porous Membrane for Gas Distribution.

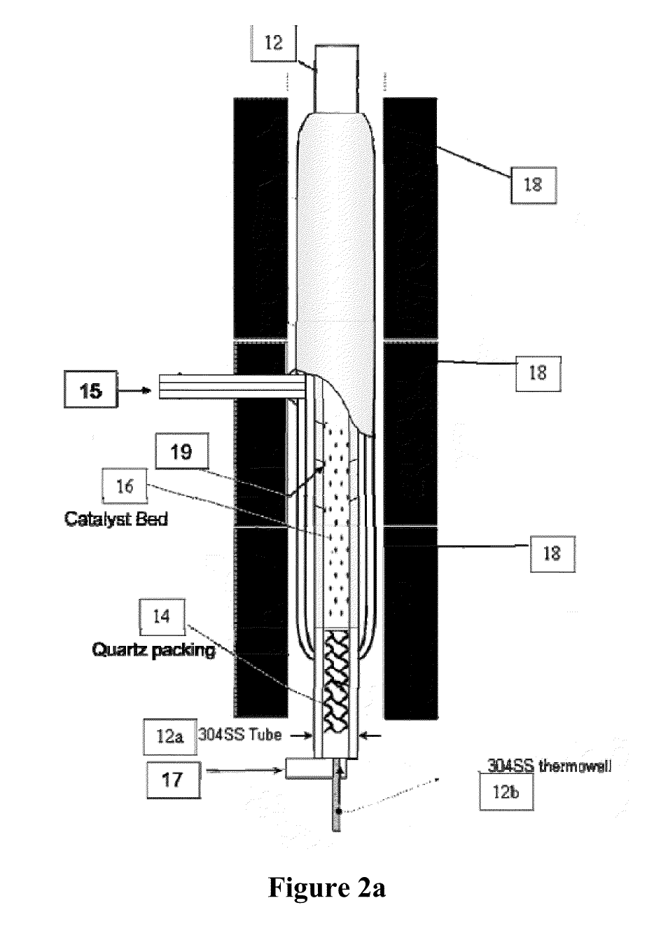

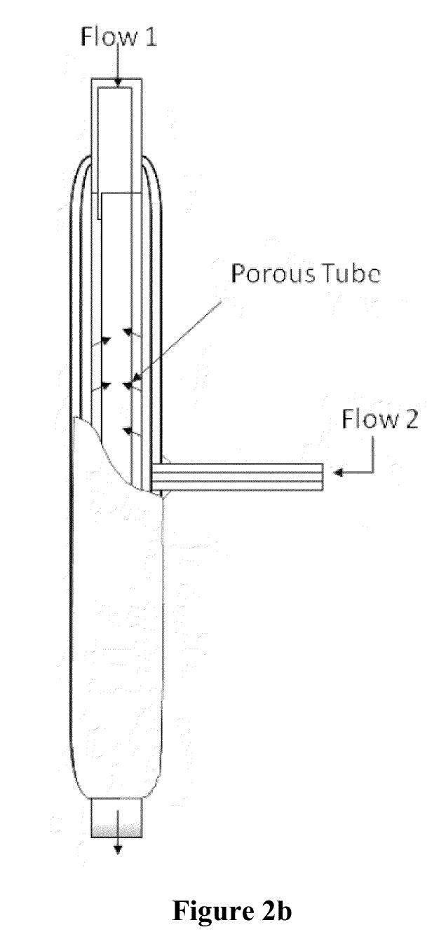

FIGS. 2a and 2b illustrate a reactor configuration that provides for uniform distribution of feed gases across the catalyst bed. In FIG. 2a, the inlet gas 17 comprises primarily methane. The inlet gas(es) 17 travel through an inert packing 14 such as quartz that supports a catalyst bed 16. A supplemental gas feed 15 consists primarily of steam, hydrogen, carbon oxides, and supplemental methane (or air/oxygen as needed) is introduced to the catalyst bed 16 by means of a porous membrane housing 19 that may be constructed from metal or ceramic materials and has average pore size ranging from about 2 microns to over 500 microns in size. The porous membrane may be made of a sintered inert metal such as stainless steel or titanium or optionally made from a ceramic. The porous membrane may also be made from a wire mesh structure. Alternatively the porous membrane is made from a porous metal. The porous membrane is constructed such that the pressure across the length of the catalyst bed does not vary significantly and the gases are introduced in a uniform and controlled manner across the length of the catalyst bed. Heating elements 18 are utilized to heat the feed gas 17 and supplemental gases 15 to the desired reaction temperatures. Processed gas consisting primarily of reaction products exit the reactor 12 and may be further processed through additional reactors or optionally organic compounds may be separated and non-organic components recycled, further processed, or used as a source of fuel. Reference numeral 12a identifies the non-porous portion of the reactor and 12b is the thermowell that is placed in an axial position in the reactor. Both 12a and 12b are preferably made of stainless steel, such as, but not limited to, Type 304 stainless steel. FIG. 2b illustrates the porous membrane structure of FIG. 2a.

For a fixed bed reactor, apparent residence time for gases in contact with the catalyst bed is 1-60,000 microseconds, more preferably 10-2000 microseconds.

Reactor with Gas Distribution Device.

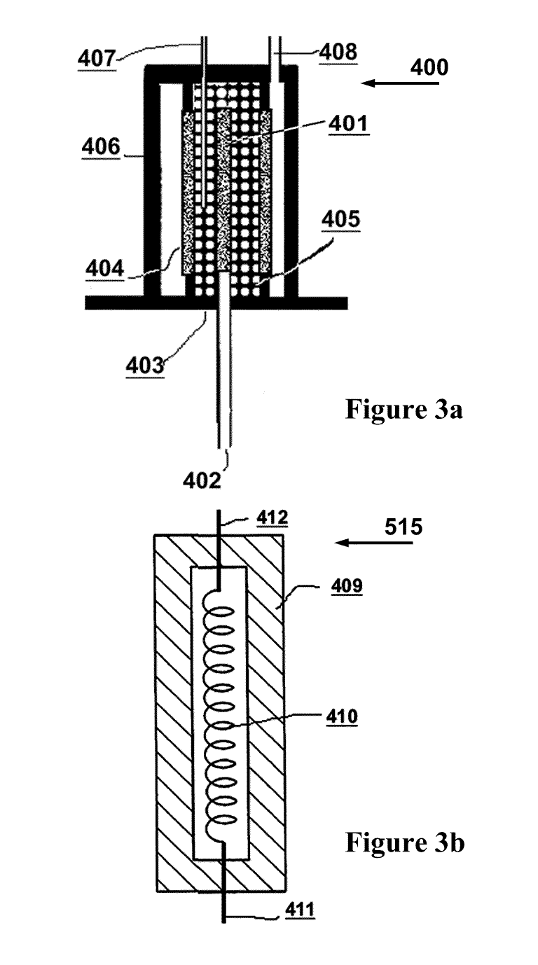

Referring to FIGS. 3a and 3b, mixed gases consisting of methane and steam with optionally added hydrogen, nitrogen, air or recycled gases enters the heating furnace 515 through a ½ inch OD stainless tube 411. The heating furnace 515 contains electric heating coils 409. The gases pass through coiled tubes 410 to obtain desired temperatures. The heated mixed gases exit the furnace 412 and enter the reactor 400.

The reactor 400 consists of an outer stainless steel tube 406. Heated gas from the heating furnace 515 enters the reactor through a ½ inch stainless steel tube 402 with a sintered/porous metal section 401 that extends for 2 inches at its termination (porous or sintered metal refers to any porous metal or ceramic material that is capable of distributing gases). The sintered/porous metal 401 helps to distribute heated gases through the catalyst bed 405. Within the catalyst bed 405 is a thermocouple 407 used to monitor and control heating. A 1½ inch diameter cylinder 404 forms an outer shell for the catalyst 405. A 2 inch section of the outer shell 404 is fitted with a sintered/porous metal section 2 inches in length and is aligned with the sintered/porous metal section of the gas feed tube 401. The porous section of the outer shell 404 may be made of porous ceramic material to reduce coking. An outer perforated metal sleeve may be placed over the porous ceramic material to supply structural integrity. Catalyzed gases exit through the sintered/porous metal section of the outer cylinder 404 before exiting the reactor through a ½ inch stainless steel tube 408. The outer shell 404 may be supplied with a means of cooling such as cooling coils or heat transfer fluid to rapidly cool the catalyzed gas prior to exiting the reactor 400 through the ½ inch stainless steel tube 408.

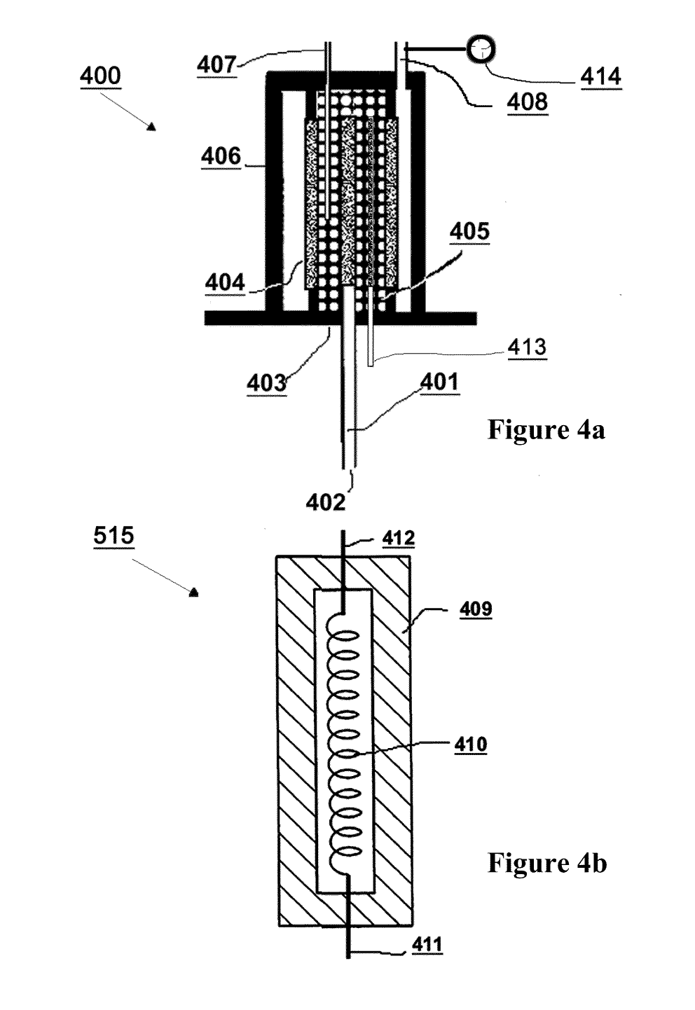

FIGS. 4a and 4b are similar to FIGS. 3a and 3b, with the addition of one or more tubes fitted with sintered/porous metal 413 that may be placed within the catalyst bed 405. Oxygen or oxygen containing gas may be introduced through the tube 413 that distributes oxygen through the catalyst bed that is used to activate the catalyst 405. One or more oxygen feed tube 413 may be placed in the catalyst bed 405 to distribute and provide oxygen for the catalytic reaction. The oxygen entering the tube 413 may be heated. One or more of the oxygen feed tubes 413 may be placed radially within the catalyst bed 405 as needed to provide the desired oxygen feed. The oxygen containing gas enters through the center sintered/porous metal tube 401 and methane (in combination with steam, recycled gas and/or nitrogen) enters through the embedded tubes within the catalyst bed 413. A pressure and temperature gauge is indicated by reference numeral 414.

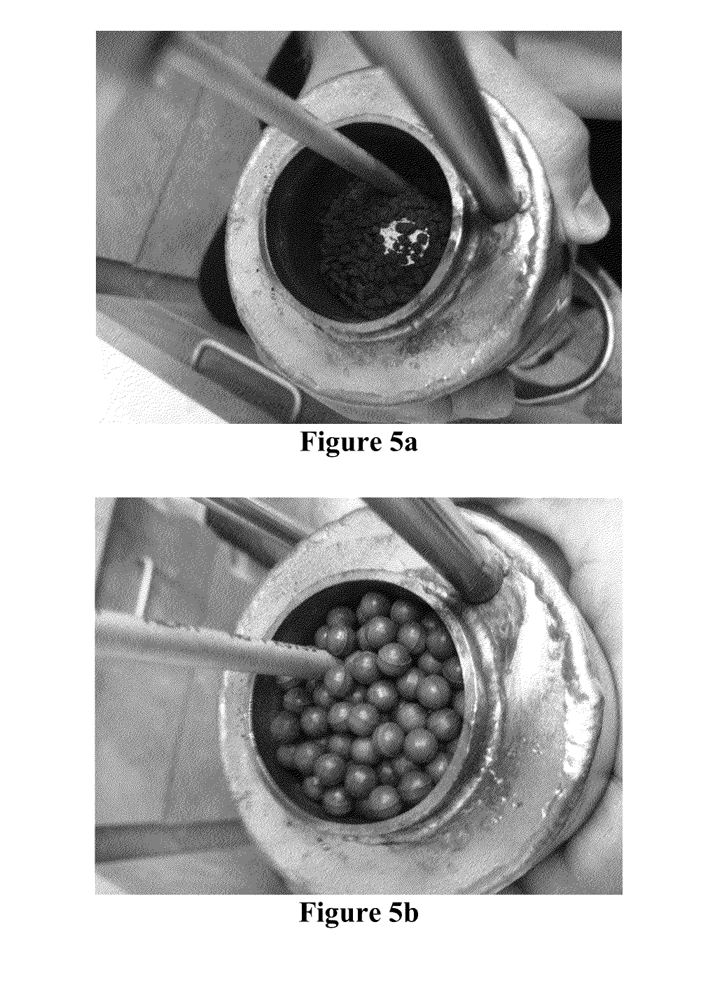

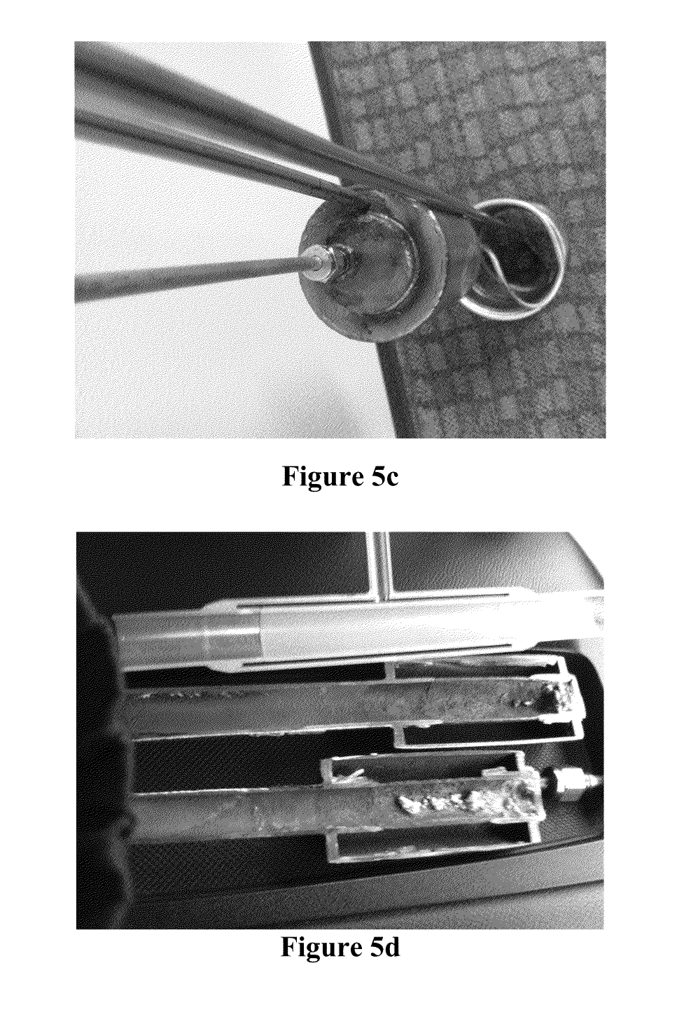

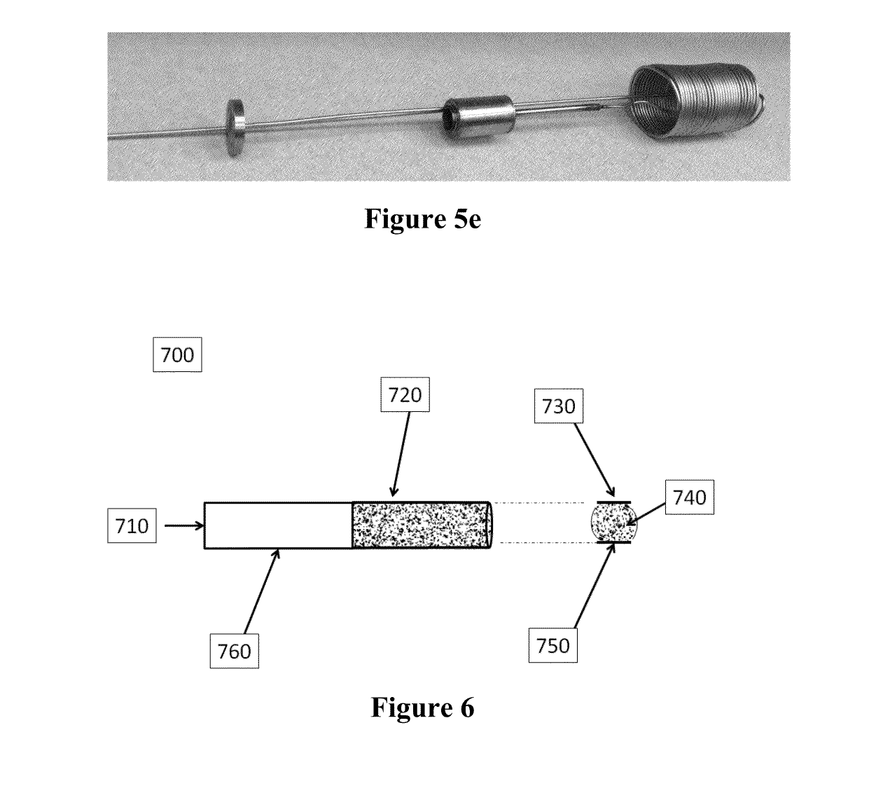

FIGS. 5a-5e illustrate exemplary reactors of the type described above in accordance with the present disclosure.

Referring to FIG. 6, an alternative design of a gas distribution device 700 is shown, which may comprise one or more spargers. Gas distribution device 700 is inserted in the catalyst bed, and capped on two sides 730 and 750 so that no gases flow through the capped sides. Gases 710 that enter the gas distribution device 700 are delivered to the sintered/porous metal section 720 and are restricted on two sides 730 and 750 from flowing axially.

Other Reactors.

The syngas and Fischer Tropsch reactor that incorporates the MF and Fischer Tropsch catalyst may be any suitable reactor or combination of reactors, such as a fixed bed reactor with axial or radial flow and with inter-stage cooling or a fluidized bed reactor equipped with internal and external heat exchangers. In some embodiments, the reactor may be operated as an adiabatic reactor. In some embodiments, a radial flow reactor (or reaction system) such as, but not limited to, a JOHNSON SCREENS® radial flow reactor vessel (http://www.johnsonscreens.com/sites/default/files/6/705/Internals%20for%20Radial%20Flow%20Reactors.pdf) may be utilized. Such a reactor is able to operate under pressurized or vacuum conditions.

In an embodiment, the reactor is a fixed bed reactor that is lined with an inert material such as alumina or fused silica or quartz. Preferably, the lining is fused quartz. In an embodiment, the process includes maintaining the catalyst and the reactant gas mixture at conversion-promoting conditions of temperature, reactant gas composition, and flow rate during a reaction period. In some embodiments, the MF is a supported catalyst. In some embodiments, the MF catalyst includes a promoter. In some embodiments, the product stream comprises one or more organic compound(s), hydrogen, steam, and carbon oxides. In some embodiments, the product stream comprises one or more organic compound(s), hydrogen, carbon monoxide, and carbon dioxide. In certain embodiments, the produced organic compounds may be largely saturated hydrocarbons due to the presence of excess hydrogen. Excess hydrogen produced as the result of steam reforming and oxidative coupling may also be recovered and used as a source of hydrogen in other chemical processes and/or energy producing processes.

Advantages.

In certain embodiments, the MF catalyst of this disclosure comprising OCM catalyst has minimal coking of the catalyst. In certain embodiments, the presence of steam reforming catalyst eliminates the need of an oxygen source, thus reducing equipment and operation costs. Furthermore, in certain embodiments, undesirable byproducts, such as carbon oxides, are minimized.

Another advantage of the catalysts and processes of this disclosure is that the resulting product mixture favors the production of hydrogen; i.e., hydrogen is a product of the present process and/or more hydrogen is combined with carbon in the final products as hydrocarbons than in conventional processes.

In some embodiments, the catalyst is incorporated into a reactor comprising a sintered/porous metal sparger (or porous membrane) (see FIGS. 2a and 2b) to distribute reactant gases evenly throughout the catalyst bed. In some cases, the porous membrane is constructed of ceramic materials, e.g., alumina, silica, titania, aluminosilicate(s), as are known in the art. In some cases, the porous membrane comprises sintered metal, e.g., titanium, stainless steel, and the like. In some cases, the porous membrane comprises porous metal.

In an embodiment, the reaction is carried out at higher than conventional temperatures with minimum carbonation and/or coking. In some embodiments, the reaction takes place at pressures higher than the atmospheric pressure. In some embodiments, the reaction takes place at pressures lower than the atmospheric pressure. In some cases, the reaction takes place at a pressure that is below atmospheric pressure or at absolute pressure of about 10 kpa absolute.

In embodiments, the method of this disclosure has higher yields compared to conventional methods that produce organic compounds. In some cases, the single pass yield of organic compounds is above 75%. In some cases, the single pass yield of organic compounds is about 75%. In some cases, the single pass yield of organic compounds is 70%-75%. In some cases, the single pass yield of organic compounds is 60%-75%. In some cases, the single pass yield of organic compounds is 50%-75%. In some cases, the single pass yield of organic compounds is 40%-75%. In some cases, the single pass yield of organic compounds is 30%-75%.

In embodiments, a reducing atmosphere of feed gases over the MF catalyst is created by addition of hydrogen to the feed gases. In embodiments a reducing atmosphere over the MF catalyst is created by generation of hydrogen by means of one of the mechanisms discussed herein.

In embodiments, the MF catalyst comprises MSR, SG and FT catalysts such that temperature equilibrium is achieved between exothermic and endothermic reactions with minimal external heat exchange required.

In embodiments, the composition of feed gases is optimized to minimize carbon oxide creation. In some embodiments, the partial pressure of each feed gas component (CO, H₂O, CO₂, CH₄, and H₂) is controlled to change the conversion and yields of the reaction products. For example, as the inlet partial pressures of CO₂ and H₂ are increased, the CO conversions decrease. In the cases of increasing H₂O partial pressure or decreasing CO partial pressure, the CO conversion increases.

In certain embodiments, different MF catalysts are employed in the two different locations within the reactors. For example, iron-based catalysts may be used for high temperature (300° C. to 900° C.) and copper-based for low temperature (150° C. to 300° C.) water gas shift reactions. The exact composition of these catalysts may vary according to their specific applications and their accompanying supports (i.e. ZnO/Al₂O₃, CeO₂, etc.). In an embodiment, the SG catalyst comprises nickel as the active component for promoting syngas production due to the resistance to sintering at elevated operating temperatures.

Mechanisms.

The mechanism disclosed herein is to produce organic compounds (e.g., alcohols and hydrocarbons) from simple alkanes, primarily methane. As used herein, the term “organic compounds” refers to compounds such as, but not limited to, ethylene, ethane, propylene, propane, butane, butene, heptane, hexane, heptene, octane, and all other linear and cyclic hydrocarbons where two or more carbons are present. “Organic compounds” also refers to alcohols, esters, and other oxygen containing organic compounds. Methanol is a single carbon molecule that is also included herein as an alcohol that is produced by the present disclosure.

The mechanism disclosed herein to produce organic compounds is by means of two or more chemical reactions occurring in a single reactor. The present invention does not require oxygen in the feed gas under normal operation conditions.

In an embodiment, a first chemical reaction involving water-gas shift reactions utilizes a reducing atmosphere and reduced catalyst (i.e. metal catalyst not containing oxygen) to produce syngas (CO and H₂) from steam and methane. The second chemical reaction in the process involves what is commonly called the Fischer Tropsch reaction where syngas is converted to organic compounds and/or simple alcohols. The methods and systems of disclosure are able to produce syngas and convert it into organic compounds (e.g., hydrocarbons and simple alcohols) within a single reactor by means of the reactor designs and catalysts as described herein.

In another embodiment, oxidative or non-oxidative dehydrogenation of methane takes place first to produce reactive species that then form organic compounds. Higher carbon number hydrocarbons may be formed with the addition of chain growing FT-type catalysts.

Without being limited by theory, the combination of OCM catalyst and MSR catalyst may produce organic compounds due to production of mobile species, i.e., adsorbed oxygen or adsorbed OH produced under the action of MSR catalyst migrate from the active sites of MSR catalyst to the OCM catalyst to create active sites for producing an adsorbed methyl species, which desorbs to produce the methyl radical. The methyl radical or even the adsorbed methyl species combines at the reaction temperatures to form adsorbed ethane which desorbs to produce ethane. The methyl radical in the gas phase may also combine to produce ethane, which will dehydrogenate to ethylene.

In embodiments, the MSR promoting catalyst component of the MF catalyst creates syngas by means of reactions that may be depicted as follows:

CH₄+H₂O [symbol] CO+3H₂ ΔH=+206 kJ/mol

CO+H₂O [symbol] CO₂+H₂ ΔH=−41 kJ/mol

The CO+H₂O [symbol] CO₂+H₂ reaction is generally referred to as the water-gas-shift (WGS) reaction. These reactions are generally carried out in conventional reactors in the temperature range of 300° C. to 900° C. in multiple adiabatic stages with inter-stage cooling to obtain higher conversions overall. Lower temperatures are generally desirable to minimize carbon formation with steam to carbon ratios ranging from about 2 to 5.

In embodiments, the SG catalyst component of the MF catalyst creates mainly syngas by means of the following reactions;

CH₄+CO₂ [symbol] 2CO+2H₂ ΔH=+165 kJ/mol

CH₄ [symbol] C+2H₂ ΔH=+75 kJ/mol

2CH₄+H₂+½H₂O→3H₂+C₂H₄+½H₂O

In an embodiment, a MF catalyst comprises a MSR catalyst and a FT catalyst. In such cases, methane is converted to CO via MSR reaction; the MSR catalyst is doped with a Fischer-Tropsch catalyst (such as cobalt and/or manganese). The mechanism is that the MSR catalyst doped with a FT catalyst performs the reforming steps by first making CO from the MSR reaction and then converts CO to hydrocarbons.

In some embodiments, under the action of the MF catalyst, the following reactions may take place:

2CH₄+H₂+½H₂O→3H₂+C₂H₄+½H₂O

CH₄+H₂O→CO+3H₂ [ΔH=+206 kJ mol⁻¹]

CO+H₂O→CO₂+H₂ [ΔH=−41 kJ mol⁻¹]

CH₄+2H₂O→CO₂+4H₂ [ΔH=+165 kJ mol⁻¹]

CH₄+CO₂ [symbol] 2CO+2H₂ [ΔH=+247 kJ=mol⁻¹]

CH₄ [symbol] C+2H₂ [ΔH=+75 kJ=mol⁻¹]

In some embodiments, metals present in the MF catalyst also produce alcohols through the following reactions:

CO+2H₂ [symbol] CH₃OH [ΔH=−90.8 kJ=mol⁻¹]

CO₂+3H₂ [symbol] CH₃OH+H₂O [ΔH=−49.6 kJ=mol⁻¹]

### EXAMPLES

### Example 1

### Catalyst Preparation

A catalyst comprising oxides of lanthanum, cobalt, sodium, tungsten, and manganese is prepared as follows. Dissolve 9 grams ammonium tungstate (99.9% purity from Sigma-Aldrich Co., St. Louis, Mo.) and 1 gram sodium hydroxide (pellets, purity 99.998%, from Sigma-Aldrich Co., St. Louis, Mo.) in 200 mL deionized water at 70° C. to about 80° C. Dissolve 27.1 grams of cobalt(II) nitrate hexahydrate (from Sigma-Aldrich Co. 99% purity) in water at about 70° C. Combine the dissolved salts and add 30 grams of manganese(IV) oxide (reagent plus purity, 99% from Aldrich). Separately dissolve 1 gram lanthanum nitrate (Laboratory grade, Fisher Scientific) in 30 mL of water and add it to the dissolved salt solution with 10 mL ammonium hydroxide (A.C.S. reagent grade from Sigma-Aldrich Co.) to achieve an alkaline pH. The mixture is heated at 80° C. for 2-3 hours and the resulting catalyst paste is placed in the oven at 120° C. and dried. The dried catalyst is calcined in a furnace that is continually purged with air during initial calcination. The catalyst is heated to 300° C. (at a rate of 15° C./min.) and held for thirty minutes at 300° C. and then increased to 550° C. at a rate of 15° C./min. and held at that temperature for 2 hours. The furnace temperature is then increased to 860° C. at the rate of 20° C./min. and held at that temperature for 24 hours. The furnace is then cooled to room temperature, and the catalyst crushed to a #40 sieve (approximately 425 microns or 0.0165 inches). To this powder is mixed 10% by weight of a reforming catalyst that is comprised of rhodium on an alumina substrate. The reforming catalyst is supplied by BASF of Florham Park, N.J., containing 5 wt % rhodium (5% RH AP 8 RD). This mixed catalyst is then pelletized in an Arbor press at 7 tons. Resulting pellets are approximately ½ inch in diameter. The pellets are then crushed to a 1-2 mm size and then annealed under inert conditions at 1000° C. for 8 hours with a heating rate of 20° C./min.

Other raw materials that may be used to produce catalyst of similar end composition include ammonium heptamolybdate [(NH₄)₆MO₇O₂₄.4H₂O], also referred to as ammonium molybdate tetrahydrate. Based upon the desired metal ratios in the end product, one skilled in the art may determine the quantities of starting materials needed to prepare a particular catalyst composition.

Silicon nitride is incorporated with the reforming catalyst or MSR catalyst if increasing fusion temperature of the catalyst is required. Three weight ratios of OCM to MSR catalyst of 99:1, 90:10, 80:20 and catalyst to Si₃N₄ of 50:50, 80:20 and 90:10 are evaluated. The resulting catalysts contain the same phases as the non-silicon nitride containing catalysts as well as silicon dioxide and silicon nitride crystalline phase.

### Example 2

### Catalyst Preparation II

A catalyst designated MR-34-18-VIII-RS2 was prepared incorporating lanthanum, cobalt, sodium, tungsten, manganese, and rhodium. The starting metal compounds were all purchased from Sigma-Aldrich Co., St. Louis, Mo. and were in the form of cobalt nitrate hydrate; lanthanum nitrate, sodium hydroxide, manganese IV oxide, ammonium tungstate, and rhodium (III) nitrate.

The following procedure was used to make the catalyst. Add 36 grams of ammonium tungstate and 4 grams of sodium hydroxide to 800 milliliters de-ionized water. In a separate container added 108.4 grams of cobalt nitrate hydrate to 200 milliliters of de-ionized water.

In a separate container dissolve 4 grams of lanthanum nitrate in 120 milliliters de-ionized water then add 40 milliliters ammonium hydroxide.

Combine the three solutions and add 120 grams of manganese IV oxide and 10 grams of rhodium nitrate. Heat the mixed solution until it forms a paste and place in oven at 212° F. for 8 hours. Once dried, the catalyst is calcined according to the following schedule: 300° C. for 15 minutes. Hold at 300° C. for an additional 30 minutes with air purge. Increase temperature over 15 minutes to 550° C. and hold for 2 hours. Raise temperature to 860° C. and hold for 24 hours.

Remove and cool catalyst and crush into granules that are then placed in a furnace for at 1000° C. for 4 hours with air flow. Catalyst net weight was 162.3 grams.

The following describes the detailed catalyst preparation procedure.

Components
1) 108.4 grams of cobalt nitrate hydrate
2) 4 grams of lanthanum nitrate
3) 40 milliliters of ammonium hydroxide
4) 4 grams of sodium hydroxide
5) 120 grams of manganese IV oxide
6) 36 grams of ammonium tungstate
7) 10 grams rhodium (III) nitrate solution
8) de-ionized water

Day 1

7:30 AM: 36 grams of ammonium tungstate and 4 grams of sodium hydroxide and 800 milliliters of de-ionized water are added to a 2500 milliliter beaker.

7:35 AM: 108.4 grams of cobalt nitrate hydrate and 200 milliliters of de-ionized water are added to a 500 milliliter beaker.

7:40 AM: 4 grams of lanthanum nitrate are dissolved in 120 milliliters of de-ionized water in a second 500 milliliter beaker and then 40 milliliters of ammonium hydroxide is added.

8:45 AM: The contents of the above three beakers are combined and thoroughly mixed to obtain a solution in a second 2500 milliliter beaker. 120 grams of manganese IV oxide and 10 grams of rhodium nitrate are added to the solution and heated until the water is evaporated and a paste is formed.

1:45 PM: The paste is put in a glass plate to dry overnight in an oven.

Day 2 (The glass plate with the paste is taken out of the oven, which contains 197.4 grams of catalyst. The catalyst is placed in a furnace for 24 hours of calcination with compressed air.)

8:00 AM: Start calcination at 300° C. and hold for 15 minutes.

8:15 AM: Start air compressor and hold at 300° C. for 30 minutes.

8:45 AM: Increase the furnace temperature over 15 minutes to 550° C.

9:00 AM: Hold the furnace temperature at 550° C. for 2 hours.

11:00 AM: Raise the furnace temperature to 860° C. and hold for 24 hours.

Day 4

7:00 AM: Remove catalyst from the furnace. The catalyst weighs 173.7 grams and is sent to be pressed into granules.

Day 7

7:00 AM: Place catalyst granules in the furnace for calcination at 1000° C. for 4 hours. The catalyst net weight is 165.6 grams.

7:05 AM: Start calcination with compressed air.

7:52 AM: The furnace temperature is at 1000° C. with compressed air on.

11:52 AM: The furnace is turned off. The catalyst net weight is 162.3 grams.

### Example 3

### Catalyst Characterization

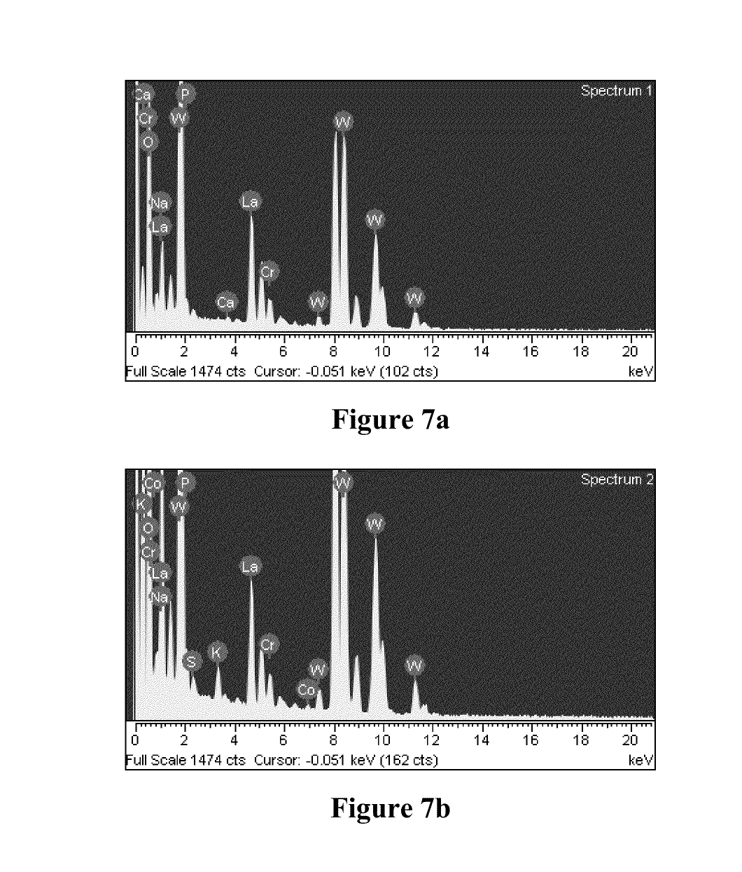

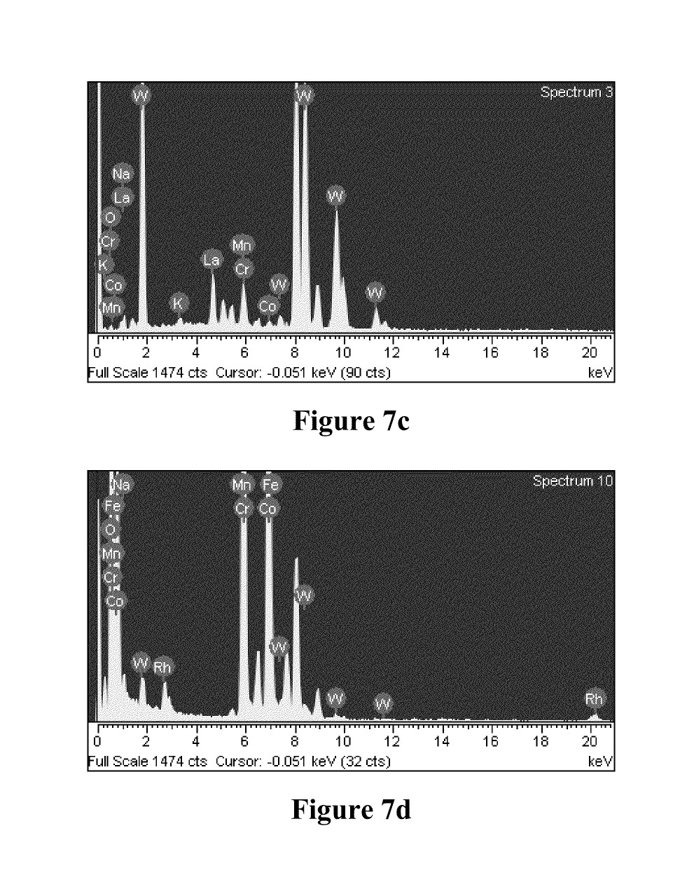

A sample catalyst prepared according to Example 2 is analyzed by energy-dispersive X-ray spectroscopy (EDS). Note that the EDS shows traces of elements that are not part of the catalyst formulation (e.g., Cr, K, P, Ca, S, Fe); these are attributable to the sample holder. Furthermore, because the catalyst is heterogeneous on the scale of the sample size analyzed via EDS, many EDS spectra (FIGS. 7a-d) would be required to obtain a quantitative determination of the composition of an entire catalyst pellet or granule. Accordingly, the EDS results provided below should not be considered quantitative analyses of a representative large sample of catalyst, but as illustrative of specific, small portions of catalyst.

The following results show that the catalyst compositions are heterogeneous. For example, Rh is disbursed on the surface of the catalyst and therefore is not present in all the spectra.

Quantitation method: Cliff Lorimer thin ratio section.
Processing option: All elements analyzed (Normalized)
Number of iterations=3; Standardless

| Spectrum 1 (Figure 7a) | | | Spectrum 2 (Figure 7b) | | |
|---|---|---|---|---|---|
| Peak possibly omitted: | | | Peaks possibly omitted: 32.970, | | |
| 33.383 keV | | | 33.410 keV | | |
| Element | Weight | Atomic | Element | Weight | Atomic |
| (orbital) | % | % | (orbital) | % | % |
| O (K) | 15.13 | 58.87 | O (K) | 17.75 | 56.93 |
| Na (K) | 3.41 | 9.23 | Na (K) | 8.44 | 18.83 |
| P (K) | 0.17 | 0.35 | P (K) | 0.10 | 0.17 |
| Ca (K) | 0.35 | 0.54 | S (K) | 0.31 | 0.50 |
| Cr (K) | 0.69 | 0.82 | K (K) | 1.01 | 1.32 |
| La (L) | 27.70 | 12.41 | Cr (K) | 0.56 | 0.56 |
| W (M) | 52.55 | 17.79 | Co (K) | 0.31 | 0.27 |
| Totals | 100.00 | | La (L) | 16.21 | 5.99 |
| | | | W (M) | 55.30 | 15.43 |
| | | | Totals | 100.00 | |
| Spectrum 3 (Figure 7c) | | | Spectrum 10 (Figure 7d) | | |
| Peak possibly omitted: | | | Peaks possibly omitted: 12.820, | | |
| 33.450 keV | | | 14.960, 25.260 keV | | |
| Element | Weight | Atomic | Element | Weight | Atomic |
| (orbital) | % | % | (orbital) | % | % |
| O (K) | 1.06 | 7.70 | O (K) | 27.75 | 58.49 |
| Na (K) | 1.12 | 5.67 | Na (K) | 0.70 | 1.03 |
| K (K) | 0.90 | 2.68 | Cr (K) | 0.43 | 0.28 |
| Cr (K) | 2.20 | 4.92 | Mn (K) | 28.23 | 17.33 |
| Mn (K) | 7.75 | 16.40 | Fe (K) | 0.55 | 0.33 |
| Co (K) | 1.52 | 3.00 | Co (K) | 36.74 | 21.02 |
| La (L) | 27.45 | 22.97 | Rh (L) | 3.43 | 1.12 |
| W (M) | 58.00 | 36.67 | W (M) | 2.16 | 0.40 |
| Totals | 100.00 | | Totals | 100.00 | |

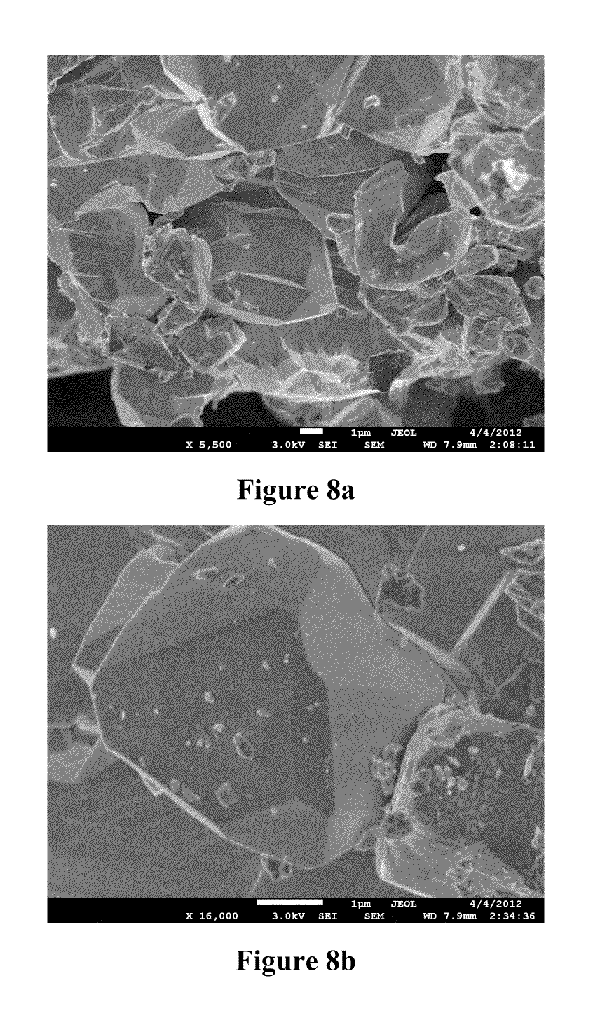

Scanning electron microscopy (SEM) is also performed on the sample catalyst with results as illustrated in the micrographs provided as FIGS. 8a and 8b. The analysis also shows the heterogeneous nature of the catalyst.

While preferred embodiments of the invention have been shown and described, modifications thereof may be made by one skilled in the art without departing from the spirit and teachings of the invention. The embodiments described herein are some only, and are not intended to be limiting. Many variations and modifications of the invention disclosed herein are possible and are within the scope of the invention. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, and so forth). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, and the like.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the preferred embodiments of the present invention. The disclosures of all patents, patent applications, and publications cited herein are hereby incorporated by reference, to the extent they provide some, procedural or other details supplementary to those set forth herein.

We claim:

1. A catalyst composition for producing organic compounds comprising
(a) a catalyst that promotes the oxidative coupling of methane (OCM) and a methane steam reforming (MSR) catalyst, wherein said catalyst composition causes oxidative dehydrogenation to form reactive species and oligomerization of said reactive species to produce said organic compounds; or
(b) a catalyst that promotes syngas generation (SG) and a Fischer-Tropsch (FT) catalyst wherein said catalyst composition causes non-oxidative dehydrogenation to form reactive species and oligomerization of said reactive species to produce said organic compounds; or
(c) an SG catalyst, an MSR catalyst, and an FT catalyst wherein said catalyst composition causes non-oxidative dehydrogenation to form reactive species and oligomerization of said reactive species to produce said organic compounds; or
(d) an FT catalyst and an MSR catalyst wherein said catalyst composition causes reforming reactions and chain growing reactions to produce said organic compounds.

2. The catalyst composition of claim 1 wherein the OCM catalyst comprises a transition metal; or a rare earth metal oxide; or a component selected from the group consisting of sodium oxide, cobalt oxide, tungsten oxide, silicon oxide, manganese oxide, and combinations thereof; or silicon nitride.

3. The catalyst composition of claim 1 wherein the MSR catalyst comprises a metal oxide wherein the metal is selected from the group consisting of cobalt (Co), iron (Fe), molybdenum (Mo), tungsten (W), cerium (Ce), rhodium (Rh), platinum (Pt), palladium (Pd), titanium (Ti), zinc (Zn), nickel (Ni), ruthenium (Ru), and combinations thereof; or wherein the MSR catalyst comprises a metal compound, wherein the metal is selected from the group consisting of nickel (Ni), cobalt (Co), iron (Fe), ruthenium (Ru), molybdenum (Mo), tungsten (W), rhodium (Rh), and combinations thereof; and wherein the MSR catalyst comprises a support material selected from the group consisting of alumina, silica, magnesia, and combinations thereof.

4. The catalyst composition (a) of claim 1 wherein the weight ratio of the OCM catalyst to the MSR catalyst is in the range of from about 50:1 to about 99:1; and optionally the catalyst composition (a) of claim 1 comprises 0.1-99 wt % of rhodium.

5. The catalyst composition of claim 1 wherein said catalyst composition maintains its catalytic activity in the temperature range of from about 300° C. to about 1200° C.

6. The catalyst composition of claim 1 further comprising a promoter.

7. The catalyst composition of claim 1 wherein the catalysts are deposited on a support to form the catalyst composition or wherein the catalysts are dry blended to form the catalyst composition.

8. A method of producing organic compounds comprising
contacting a reactant gas mixture comprising natural gas and steam and optionally hydrogen with a catalyst composition, wherein the catalyst composition comprises
(a) a catalyst that promotes the oxidative coupling of methane (OCM) and a methane steam reforming (MSR) catalyst, wherein said catalyst composition causes oxidative dehydrogenation to form reactive species and oligomerization of said reactive species to produce said organic compounds; or
(b) a catalyst that promotes syngas generation (SG) and a Fischer-Tropsch (FT) catalyst wherein said catalyst composition causes non-oxidative dehydrogenation to form reactive species and oligomerization of said reactive species to produce said organic compounds; or
(c) an SG catalyst, an MSR catalyst, and an FT catalyst wherein said catalyst composition causes non-oxidative dehydrogenation to form reactive species and oligomerization of said reactive species to produce said organic compounds; or
(d) an FT catalyst and an MSR catalyst wherein said catalyst composition causes reforming reactions and chain growing reactions to produce said organic compounds.

9. The method of claim 8 further comprising preheating the steam.

10. The method of claim 9 wherein the steam is distributed over the catalyst composition through a permeable membrane or a sintered metal tube.

11. The method of claim 8 wherein the single pass yield of organic compounds is no less than 75%.

12. A gas exchanger comprising
a gas seal separating the exchanger into at least two compartments wherein said seal prevents gas exchange between said compartments; and
a catalyst.

13. The gas exchanger of claim 12 wherein said exchanger is a rotary gas exchanger, optionally driven by a mechanical drive configured to rotate the exchanger in either a clockwise or counterclockwise direction.

14. The gas exchanger of claim 12 wherein said gas seal is configured to withstand a temperature of up to 900° C.

15. The gas exchanger of claim 12 further comprising an outer shell, configured to contain said catalyst; and optionally wherein said catalyst is in a bed formation.

16. The gas exchanger of claim 15 wherein said catalyst bed is configured to cause turbulent gas flow and to provide minimal pressure drop across the catalyst bed.

17. The gas exchanger of claim 12 wherein said catalyst is coated on ceramic or metal surfaces used to pack the exchanger.

18. A method comprising
converting a reactant into a product in a rotating reactor,
exchanging heat between the reactant and the product,
promoting the reaction with a catalyst, and
re-activating the catalyst;
wherein the reactor comprises a rotary gas heat exchanger comprising said catalyst and a gas seal separating the exchanger into at least two compartments, and wherein said seal prevents gas exchange between said compartments.

19. The method of claim 18 wherein the reactor comprises an individual rotary gas heat exchanger; or multiple stacked rotary gas heat exchangers and optionally inter-exchanger heat transfer or gas injection.

20. The method of claim 18 further comprising rotating the reactor at a speed sufficient to provide sufficient residence time for the reactant to be converted into the product; and optionally rotating the reactor at a speed sufficient to provide sufficient residence time for the catalyst to be re-activated.
